(19) United States
(12) Patent Application Publication
(10) Pub. No.: US 2023/0096615 A1
(43) Pub. Date: Mar. 30, 2023

(54) METALLIC ORTHOPAEDIC IMPLANT AND METHOD OF MAKING THE SAME

(71) Applicant: DePuy Synthes Products, Inc., Raynham, MA (US)

(72) Inventors: Oscar A. QUINTANA-PONCE, Warsaw, IN (US); Bryan J. SMITH, Warsaw, IN (US)

(21) Appl. No.: 17/800,108

(22) PCT Filed: Feb. 19, 2021
(86) PCT No.: PCT/IB2021/051427

§ 371 Date: Aug. 16, 2022

Related U.S. Application Data
(60) Provisional application No. 62/978,532, filed on Feb. 19, 2020.

(51) Int. Cl. A61F 2/30; A61F 2/38

(52) U.S. Cl. CPC A61F 2/3094; A61F 2/3859; A61F 2002/3092; A61F 2310/00023; A61F 2310/00485; A61F 2310/00491

(57) ABSTRACT

An orthopaedic implant includes a femoral component having a metallic zirconium and niobium coating disposed therein. A method of making the femoral component using direct energy deposition or co-molding is also disclosed.

This application claims priority to U.S. Patent App. Ser. No. 62/978,532, filed on Feb. 19, 2020, which is expressly incorporated herein by reference.

### TECHNICAL FIELD

The present disclosure relates generally to an orthopaedic prosthesis and more particularly to an orthopedic prosthesis having a metallic component.

### BACKGROUND

Joint arthroplasty is a well-known surgical procedure by which a diseased and/or damaged natural joint is replaced by a prosthetic joint. A typical knee prosthesis includes a patella prosthetic component, a tibial tray, a femoral component, and a tibial bearing positioned between the tibial tray and the femoral component. Femoral components are designed to be attached to a surgically-prepared distal end of a patient's femur. Tibial trays are designed to be attached to a surgically-prepared proximal end of a patient's tibia.

The femoral component and the tibial component can be made of biocompatible materials such as metal alloys of cobalt-chrome or titanium. The tibial bearing component disposed between the femoral component and the tibial tray can be formed from a plastic material like polyethylene. However, cobalt alloys tend to be expensive. Accordingly, a need exists for a component made from a non-cobalt metal material and a method of forming the same. For example, a need exists for a femoral component of a knee prosthesis out of a non-cobalt metal material and a method for forming the same.

### SUMMARY

In some embodiments, an orthopaedic knee prosthesis is disclosed. The knee prosthesis includes a femoral component configured to be coupled to a distal end of a patient's femur. The femoral component comprises a substrate comprising a titanium alloy. The substrate comprises a condylar surface and a bone-facing surface opposite the condylar surface. Further, the knee prosthesis includes an articular layer. The articular layer comprises an alloy of zirconium and niobium and is disposed upon the condylar surface of the substrate. A portion of the zirconium in the alloy of the articular layer can be oxidized, if desired, to generate a zirconium oxide layer having a thickness of, for example, about 2 micrometers to about 5 micrometers as measured from the exposed, outer surface of the of the articular layer. As discussed in greater detail below, the percentage by weight of zirconium in the articular layer can vary. For example, percentage by weight of zirconium can be from about 93% to about 98%. In addition, the percentage by weight of niobium in the articular layer can also vary. For example, the percentage by weight of niobium can be from about 2% to about 7%. The bone-facing surface may have a bone-engaging layer disposed thereon.

In some embodiments, a method of forming a femoral component is disclosed. The method includes utilizing direct energy deposition to form an articular layer on to the condylar surface of the femoral component. The femoral component comprises a substrate comprising an alloy of titanium. In some embodiments, the articular layer comprises a zirconium alloy. In some embodiments, the articular layer comprises an alloy of zirconium and niobium. In some embodiments, the method further includes oxidizing a portion of the articular layer disposed upon the condylar surface of the femoral component to generate a layer of zirconium oxide. The percentage by weight of zirconium and niobium can vary as indicated above.

In some embodiments, a method of forming a femoral component by metal injection molding (MIM) is disclosed. The method includes injecting a first mixture comprising a zirconium alloy and a binder agent into a first mold to form an articular layer; and injecting a second mixture comprising a titanium alloy and a binder agent into a second mold to form a substrate, sintering the articular layer and substrate together, and oxidizing a portion of the articular layer. In some embodiments, the substrate comprises an alloy of titanium. In some embodiments, the articular layer comprises an alloy of zirconium.

### BRIEF DESCRIPTION OF THE DRAWINGS

The detailed description particularly refers to the following figures, in which:

FIG. 1 is an exploded perspective view of an orthopaedic knee prosthesis;

FIG. 2 is a cross-sectional view of the femoral component and tibial bearing of FIG. 1 along the sagittal plane, taken generally along line 2-2 of FIG. 1, as viewed in the direction of the arrows;

FIG. 3 is a flow chart illustrating exemplary steps for forming the femoral component using direct energy deposition; and

FIG. 4 is a flow chart illustrating exemplary steps for forming the femoral component using a co-molding process.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

Terms representing anatomical references, such as anterior, posterior, medial, lateral, superior, inferior, etcetera, may be used throughout the specification in reference to the orthopaedic implants or orthopaedic prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

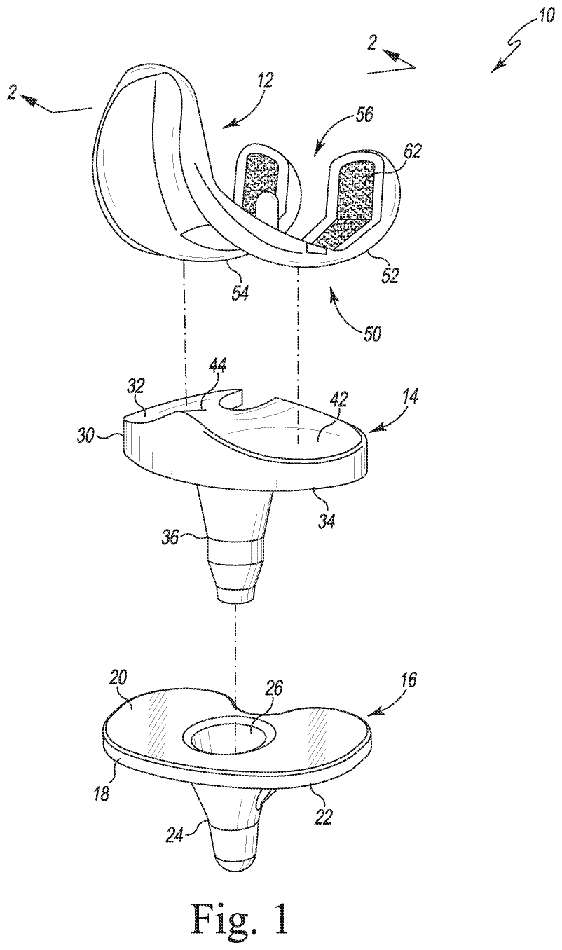

Referring now to FIG. 1, in one embodiment, an orthopaedic knee prosthesis 10 includes a femoral component 12, a tibial bearing 14, and a tibial tray 16. The femoral component 12 is configured to articulate with the tibial bearing 14, which is configured to be coupled with the tibial tray 16. In the illustrative embodiment of FIG. 1, the tibial bearing 14 is embodied as a rotating or mobile tibial bearing and is configured to rotate relative to the tibial tray 16 during use. However, in other embodiments, the tibial bearing 14 may be embodied as a fixed tibial bearing, which may be limited or restricted from rotating relative the tibial tray 16.

The tibial tray 16 is configured to be secured to a surgically-prepared proximal end of a patient's tibia (not shown). The tibial tray 16 may be secured to the patient's tibia via use of bone cement or other attachment methods. The tibial tray 16 includes a platform 18 having a top surface 20 and a bottom surface 22. Illustratively, the top surface 20 is generally planar. The tibial tray 16 also includes a stem 24 extending downwardly from the bottom surface 22 of the platform 18. A cavity or bore 26 is defined in the top surface 20 of the platform 18 and extends downwardly into the stem 24. The bore 26 is formed to receive a complimentary stem of the tibial bearing 14 as discussed in more detail below.

As discussed above, the tibial bearing 14 is configured to be coupled with the tibial tray 16. The tibial bearing 14 includes a platform 30 having an upper bearing surface 32 and a bottom surface 34. In the illustrative embodiment wherein the tibial bearing 14 is embodied as a rotating or mobile tibial bearing, the bearing 14 includes a stem 36 extending downwardly from the bottom surface 34 of the platform 30. When the tibial bearing 14 is coupled to the tibial tray 16, the stem 36 is received in the bore 26 of the tibial tray 16. In use, the tibial bearing 14 is configured to rotate about an axis defined by the stem 36 relative to the tibial tray 16. In embodiments wherein the tibial bearing 14 is embodied as a fixed tibial bearing, the bearing 14 may or may not include the stem 36 and/or may include other devices or features to secure the tibial bearing 14 to the tibial tray 16 in a non-rotating configuration.

The upper bearing surface 32 of the tibial bearing 14 includes a medial bearing surface 42 and a lateral bearing surface 44. The medial and lateral bearing surfaces 42, 44 are configured to receive or otherwise contact corresponding medial and lateral condyles of the femoral component 12 as discussed in more detail below. As such, each of the bearing surface 42, 44 has a concave contour.

The femoral component 12 is configured to be coupled to a surgically-prepared surface of the distal end of a patient's femur (not shown). The femoral component 12 may be secured to the patient's femur via use of bone adhesive or other attachment methods. The femoral component 12 includes an outer articular surface 50 having a pair of medial and lateral condyles 52, 54. The condyles 52, 54 are spaced apart to define an intracondyle notch 56 therebetween. In use, the condyles 52, 54 replace the natural condyles of the patient's femur and are configured to articulate on the corresponding bearing surfaces 42, 44 of the platform 30 of the tibial bearing 14.

The illustrative orthopaedic knee prosthesis 10 of FIG. 1 is embodied as a posterior cruciate-retaining knee prosthesis. That is, the femoral component 12 is embodied as a posterior cruciate-retaining knee prosthesis and the tibial bearing 14 is embodied as a posterior cruciate-retaining tibial bearing 14. However, in other embodiments, the orthopaedic knee prosthesis 10 may be embodied as a posterior cruciate-sacrificing knee prosthesis.

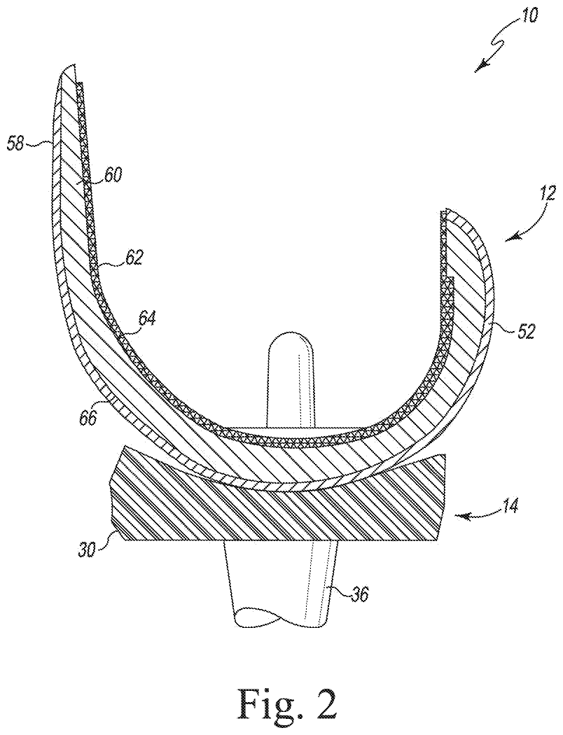

Referring now to FIGS. 1 and 2, the femoral component 12 is configured to articulate on the tibial bearing 14 during use. Each condyle 52, 54 of the femoral component 12 includes a condylar surface 66, which is convexly curved in the sagittal plane and configured to face the respective bearing surface 42, 44 of the tibial bearing 14.

As shown in FIG. 2, the femoral component 12 includes a substrate 60 and an articular layer 58. Illustratively, the articular layer 58 is disposed on the substrate 60 and is configured to interact with the tibial bearing 14. In some embodiments, the femoral component 12 includes a bone-engaging layer 62 located opposite the articular layer 58 to locate the substrate 60 therebetween. The bone-engaging layer 62 is configured to interact with a surgically prepared femur of a patient.

The substrate 60 comprises a condylar surface 66 and a bone-facing surface 64 opposite the condylar surface 66. The articular layer 58 is disposed on the condylar surface 66 of the substrate 60. A bone-engaging layer 62 may be disposed on the bone-facing surface 64. The bone-engaging layer 62 is located opposite the articular layer 58 to locate the substrate 60 therebetween. The articular layer 58 may be disposed on the condylar surface 66 by direct energy deposition or co-molded using metal injection co-molding processes as discussed further below.

In illustrative embodiments, the femoral component 12 comprising a substrate 60 and an articular layer 58 has a thickness of about 3 mm to about 8 mm. In illustrative embodiments, the femoral component 12 has thickness of about 5 mm. In some embodiments, the thickness of the substrate 60 varies from posterior to anterior regions.

In some embodiments, the femoral component 12 is configured to attach to the distal end of a patient's femur without cement. Illustratively, the femoral component 12 comprises a bone-engaging layer 62 disposed on the bone-facing surface 64. In some embodiments, the bone-engaging layer 62 comprises titanium. It should be appreciated that the bone-engaging layer 62 could be a separately-applied coating such as Porocoat® Porous Coating, which is commercially available from DePuy Synthes of Warsaw, Ind.

In some embodiments, the bone-engaging layer 62 can be defined by a porous three-dimensional structure that can include a plurality of connected unit cells. Each unit cell can define a unit cell structure that includes a plurality of lattice struts that define an outer geometric structure and a plurality of internal struts that define a plurality of internal geometric structures that are disposed within the outer geometric structure. In one example, the outer geometric structure may be a rhombic dodecahedron, and the inner geometric structures may be a rhombic trigonal trapezohedron. It should be appreciated that such geometric structures may vary to fit the needs of a given design. Further, it should be appreciated that the unit cells that make up the bone-engaging layer 62 may also have any suitable alternative geometry to fit the needs of a given design.

The bone-engaging layer 62 is formed from a metal powder. Illustratively, the metal powders may include, but are not limited to, titanium, titanium alloys, stainless steel, cobalt chrome alloys, tantalum, or niobium powders. The bone-engaging layer 62 has a porosity suitable to facilitate bony ingrowth into the bone-engaging layer 62 of the femoral component 12 when implanted into the surgically-prepared surface of the distal end of a patient's femur.

In the illustrative embodiment described herein, the bone-engaging layer 62 is additively manufactured directly onto the bone-facing surface 64 of the femoral component 12. In such an embodiment, the two structures—i.e., the femoral component 12 and bone-engaging layer 62—may be manufactured contemporaneously during a common additive manufacturing process. For example, the two structures may be manufactured contemporaneously in a single 3D printing operation that yields a common, monolithic metallic component including both structures. Alternatively, the bone-engaging layer 62 could be manufactured as a separate component that is secured to the bone-facing surface 64 of the femoral component 12.

In alternative embodiments, the femoral component 12 is configured to attach to the surgically-prepared distal end of a patient's femur using bone adhesives. In some embodiments, the bone adhesive is disposed on the bone-facing surface 64. In some embodiments, the bone-facing surface 64 is configured to receive a bone adhesive. In some embodiments, the femoral component 12 comprises a cement reservoir disposed on the bone-facing surface 64 (not shown). In some embodiments, the bone adhesive comprises bone cement.

In some embodiments, the substrate 60 comprises a metal alloy. In some embodiments, the metal alloy comprises a titanium alloy. In some embodiments, the substrate 60 comprises titanium and aluminum. In some embodiments, the substrate 60 comprises titanium and vanadium. In some embodiments, the substrate 60 comprises titanium, aluminum, and vanadium. In some embodiments, the substrate 60 comprises Ti-6Al-4V. In some embodiments, the substrate 60 consists essentially of Ti-6Al-4V.

Referring to FIG. 2, the articular layer 58 is disposed on the condylar surface 66. The articular layer 58 is located opposite the bone-facing surface 64 to locate the substrate 60 therebetween. The articular layer 58 is configured to interact with the bearing surfaces 42, 44 and to articulate with the tibial bearing 14.

The articular layer 58 comprises a metallic alloy, wherein a portion of the metallic alloy is oxidized. The oxidized portion of the articular layer 58 forms the outer articular surface 50 of the femoral component 12. In some embodiments, the articular layer 58 comprises a zirconium alloy. In some embodiments, the articular layer 58 comprises an alloy of zirconium and niobium. In some embodiments, the articular layer 58 comprises at least about 97.5% zirconium. In some embodiments, the articular layer 58 comprises at least about 2.5% niobium. In some embodiments, the articular layer 58 comprises at least about 95% zirconium and niobium alloy prior to oxidizing a portion of the articular layer 58. In some embodiments, the articular layer 58 comprises Zr-2.5Nb. In some embodiments, the articular layer 58 consists essentially of Zr-2.5Nb.

In some embodiments, the articular layer 58 comprises a percentage by weight (weight % or %) of zirconium. In some embodiments, the articular layer 58 comprises a range of zirconium of about 93% to about 99%, about 94% to about 98%, about 95% to about 97%, or about 96%. In some embodiments, the articular layer 58 comprises at least about 93%, at least about 94%, at least about 95%, at least about 96%, at least about 97%, or at least about 98% zirconium. In some embodiments, the articular layer 58 comprises about 95%, about 95.5%, about 96%, about 96.5%, about 97%, about 97.5%, about 98%, about 98.5%, or about 99% zirconium. In some embodiments, the articular layer 58 comprises up to about 93%, about 93.5%, about 94%, about 94.5%, about 95%, about 95.5%, about 96%, about 96.5%, about 97%, about 97.5%, about 98%, about 98.5%, or about 99% zirconium. In some embodiments, the articular layer 58 comprises about 97.5% zirconium. In some embodiments, the articular layer 58 comprises about 97.5% by weight zirconium prior to oxidizing the articular layer 58.

In some embodiments, the articular layer 58 comprises a percentage by weight of niobium. In some embodiments, the articular layer 58 comprises a range of niobium of about 1% to about 7%, about 2% to about 6%, about 3% to about 5%, or about 4%. In some embodiments, the articular layer 58 comprises at least about 1%, about 2%, about 3%, about 4%, about 5%, about 6%, or about 7% niobium. In some embodiments, the articular layer 58 comprises up to about 1%, about 1.5%, about 2%, about 2.5%, about 3%, about 3.5%, about 4%, about 4.5%, about 5%, about 5.5%, about 6%, about 6.5%, or about 7%. In some embodiments, the articular layer 58 comprises 2.5% niobium. In some embodiments, the articular layer 58 comprises 2.5% niobium prior to oxidizing the articular layer 58.

In some embodiments, the articular layer 58 comprises a percentage by weight of an alloy of zirconium and niobium. In some embodiments, the articular layer 58 comprises about 93% zirconium and about 7% niobium, about 94% zirconium and about 6% niobium, about 95% zirconium and about 5% niobium, about 96% zirconium and about 4% niobium, about 97% zirconium and about 3% niobium, about 98% zirconium and about 2% niobium, or about 99% zirconium and about 1% niobium. In some embodiments, the articular layer 58 comprises an alloy of about 97.5% zirconium and about 2.5% niobium denoted Zr-2.5Nb. In some embodiments, the articular layer 58 consists essentially of 97.5% zirconium and 2.5% niobium prior to oxidizing the articular layer 58.

In some embodiments, the articular layer 58 has a thickness of about 2 mm to about 5 mm. In some embodiments, the articular layer 58 has a thickness of about 2 mm to about 3 mm. In some embodiments, the articular layer has a thickness of at least about 2 mm, at least about 3 mm, at least about 4 mm, at least about 5 mm, or at least about 6 mm.

The exposed, outer articular surface 50 of the articular layer 58 is oxidized. The step of oxidizing the outer articular surface 50 of the articular layer 58 is discussed in further detail below. The step of oxidizing generates a metallic oxide layer on a portion of the articular layer 58. In some embodiments, the oxidized portion of the articular layer 58 comprises zirconium oxide (ZrO₂).

In an illustrative embodiment, the ZrO₂ layer has a thickness of about 2 μm to about 5 μm. In some embodiments, the ZrO₂ layer has a thickness of about 2 μm to about 3 μm. In some embodiments, the ZrO₂ layer has a thickness of at least about 1 μm, at least about 2 μm, at least about 3 μm, at least about 4 μm, at least about 5 μm, or at least about 6 μm.

In some embodiments, a method of forming a femoral component 12 is provided. In some embodiments, the articular layer 58 is disposed on the substrate 60 by direct energy deposition. In an exemplary embodiment, a commercially available instrument such as a LENS® provided by Optomec®) may be used to perform the direct energy deposition process. The process of direct energy deposition is generally known to those skilled in the art of metallurgy.

In an illustrative embodiment, the substrate 60 is manufactured by a means known to those in the art. For example, the substrate 60 may be manufactured by forging, casting, molding or another process known to those skilled in the art.

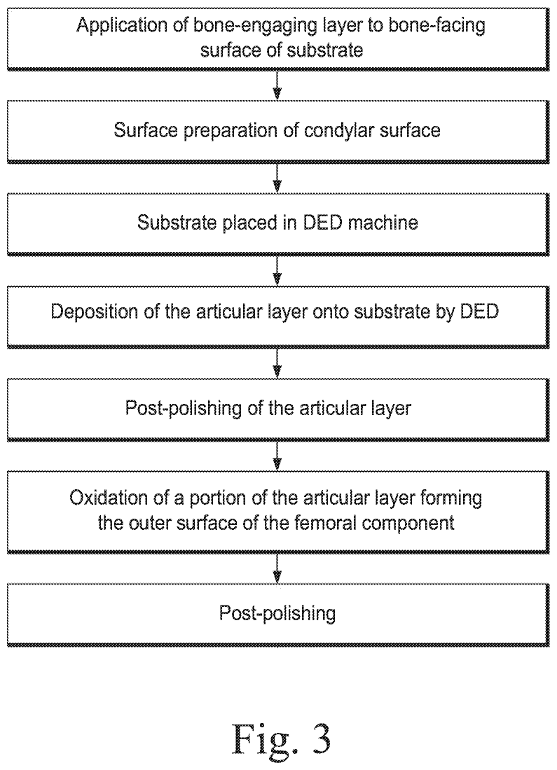

Referring to FIG. 3 for an illustrative diagram of the method, a bone-engaging layer 62 is deposited on the bone-facing surface 64 of the substrate 60. This process may be achieved using methods known to those skilled in the art. In an exemplary embodiment, the bone-facing surface 64 of the substrate 60 is roughened by blasting or using an abrasive material. Thereafter, a coating of titanium hydride particles of the desired thickness is formed on the roughened bone-facing surface 64. The titanium hydride particle coating may be formed by spraying the roughened bone-facing surface 64 with a binder and then suspending the substrate 60 in a fluidized bed of titanium hydride particles of the desired particle size distribution to form the coating by adherence of the particles to the binder. The substrate 60 is withdrawn from the fluidized bed and the binder allowed to dry.

In an alternative procedure, the titanium hydride particles may be mixed with a binder to form a viscous slurry which then is spray applied to the roughened bone-facing surface 64 to form the bone-engaging layer 62 thereon, and the bone-engaging layer 62 then is allowed to dry. Those skilled in the art will recognize other methods of generating the bone-engaging layer 62.

For further disclosure about forming a bone-engaging layer 62, U.S. Pat. No. 4,206,516 is incorporated herein by reference in its entirety.

The condylar surface 66 is prepared by blasting with particles to roughen the surface in preparation of the direct energy deposition of the articular layer 58. In an illustrative embodiment, alumina bead or grit media may be used to roughen the surface. Those of ordinary skill in the art will recognize that other materials may be used to roughen the surface prior to direct energy deposition.

The articular layer 58 is formed upon the condylar surface 66 of the substrate 60 utilizing direct energy deposition. In particular, as shown in FIG. 3, substrate 60 can be positioned in a direct energy deposition machine. The atmosphere the substrate 60 is exposed to during the direct energy deposition process is a mixture of argon and oxygen, wherein the oxygen is present at about less than 50 ppm.

During the direct energy deposition process, an atomized powdered alloy of zirconium and niobium (e.g., Zr-2.5Nb) is directed onto the condylar surface 66 and melted with a laser. The desired ratio (weight present) of zirconium to niobium for the articular layer 58 can initially be in a pre-atomized or atomized powdered form. The desired powders or bars are commercially available from ATI Specialty Alloys and Components or Materion. With respect to the laser, it can be powered between about 350 W to about 450 W. The laser scan velocity across the condylar surface 66 is about 8 mm/s to about 12 mm/s. The atomized powder is fed at a rate of about 17 gams/min to about 21 grams/min during deposition to form the articular layer 58.

In some embodiments, the formed articular layer 58 ranges in thickness from about 2 mm to about 3 mm. Still referring to FIG. 3, after the articular layer 58 is disposed on the condylar surface 66 by direct energy deposition to form the femoral component 12, the femoral component 12 undergoes a post machining and polishing process performed by methods known to those skilled in the art. In exemplary embodiments, the post machining and post polishing can be done by hand or mass finishing in a rotating tumbler with polishing media.

In some embodiments, prior to the step of oxidizing, the articular layer 58 is further processed. In some embodiments, the articular layer 58 is subjected to an abrasive surface preparation process that includes but is not limited to, grinding, buffing, mass finishing, and vibratory finishing. The further processing step is used to induce an altered surface roughness to generate a uniform oxidized metallic layer.

Next, the exposed, outer articular surface 50 of the articular layer 58 is oxidized. In some embodiments, the step of oxidation follows Kemp's process to produce a ZrO₂/Zr-2.5Nb articular layer 58. In particular, the femoral component 12 is heated to between about 500° C. to about 550° C. for about three hours to about five hours under an atmosphere of argon and oxygen in a fluidized bed. In some embodiments, the atmosphere in the fluidized bed comprises about 1% to about 3% oxygen and about 97% to about 99% argon. In some embodiments, the oxygen comprises 2.5% of total gas volume in the fluidized bed. In some embodiments, the femoral component 12 is heated to between 550° C. to about 650° C. for about five hours to about six hours under an atmosphere of argon and oxygen in a fluidized bed. In some embodiments, inert oxide granules are used in the oxidizing process. In some embodiments, the inert oxide granules comprise ZrO₂. In some embodiments, the inert oxide granules comprise alumina oxide particles.

The step of oxidizing oxidizes a portion of the zirconium in the articular layer 58 to generate a layer of ZrO₂ having a thickness of about 4 μm to about 6 μm. In particular, the thickness of the ZrO₂ can be about 5 μm thick measure from the outer articular surface 50. The ZrO₂ layer can be uniformly dispersed over the outer articular surface 50 of the articular layer 58, if desired.

For further disclosure on the procedure of oxidizing see U.S. Pat. Nos. 6,447,550 and 5,324,009 both of which are incorporated by reference in their entirety.

In some embodiments, the femoral component 12 undergoes a post-polishing procedure using methods and materials known to those skilled in the art.

In some embodiments, the femoral component 12 can be formed by co-molding the articular layer 58 and the substrate 60 using metal injection molding (MIM).

In some embodiments, the femoral component 12 can be formed by co-molding, the articular layer 58 (Zr-2.5Nb), the substrate 60 (Ti-6Al-4V), and the bone-engaging layer 62 using MIM.

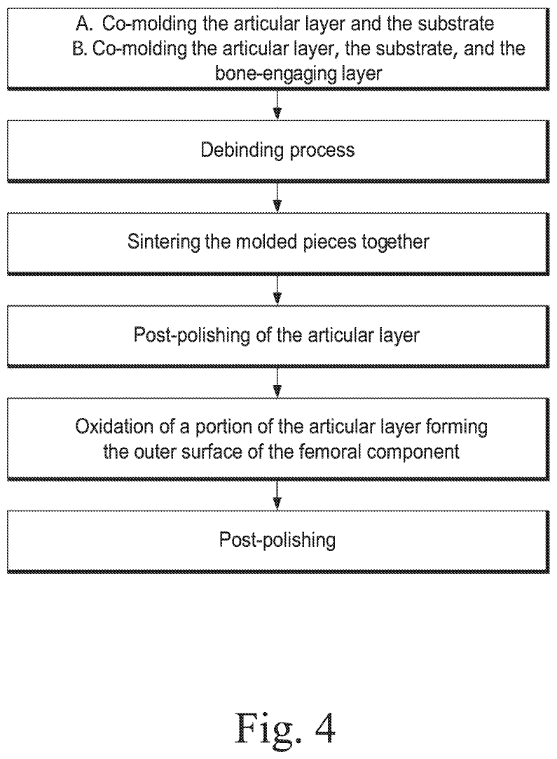

Referring to FIG. 4, in an illustrative embodiment, the femoral component 12 is formed by preparing a first mixture of powdered zirconium alloy and binder. In some embodiments, the first mixture comprises a metallic powder comprising zirconium and niobium admixed with a binder. In some embodiments, the first mixture comprises a metallic powder of Zr-2.5Nb alloy and a binder. The starting materials (also referred to as the “Feedstock”) may be purchased from a commercial vendor as a powder already mixed with the desired ratio of zirconium and niobium. In some embodiments, the first mixture may be generated by mixing a specified ratio of powdered zirconium and powdered niobium together. In some embodiments, the particle size of the metal powders is between about 0.10 μm to about 45 μm in diameter. In some embodiments, the metal powder is separated using a mesh. In an exemplary embodiment, the mesh is a 325 mesh.

The binder can be selected from a wide variety of known binder materials, including, for example, waxes, polyolefins such as polyethylenes and polyproplyenes, polystyrenes, polyvinyl chloride, polyethylene carbonate, polyethylene glycol and microcrystalline wax. The particular binder will be selected on the basis of compatibility with powder components, and ease of mixing, molding and debinding. Other considerations in selecting a binder include toxicity, shelf life, strength, lubricity, biostability, and recyclability. The polymeric binder volume fraction ranges from about 35% to about 45%.

A second mixture is generated by mixing together or purchasing a set amount of metallic powder comprising a titanium alloy and a binder. In some embodiments, the second mixture comprises a powder of a titanium alloy comprising Ti-6Al-4V and a binder. The particle size of the powders should be between about 0.10 μm to about 45 μm in diameter. The particle size can be controlled by passing the metal powder through a 325 mesh. The polymeric binder used in the first and second mixture may be the same. In some embodiments, the polymeric binder used in the first mixture is different than the polymeric binder used in the second mixture. The binder volume fraction in the second mixture is about 35% to about 45%.

In one embodiment, the method comprises injecting a first mixture into a cold mold form configured to be the shape of the articular layer 58, and injecting a second mixture into a second cold mold form configured to be the shape of the substrate 60.

In another embodiment, a third mixture comprising pure titanium or titanium alloy powder, a particle mixture, and a polymeric binder is injected into a third cold mold form configured to be the shape of the bone-engaging layer 62. The particle mixture comprises particles configured to generate pores in the bone-engaging layer 62 when removed. In one embodiments, the particles are a salt. In some embodiments, the pore size generated in the bone-engaging layer 62 is suitable to facilitate bony ingrowth into the bone-engaging layer 62 of the femoral component 12 when implanted into the surgically-prepared surface of the distal end of a patient's femur.

The three cold mold forms combine together to co-mold the articular layer 58, the substrate 60, and the bone-engaging layer 62, wherein the substrate 60 interconnects the articular layer 58 and the bone-engaging layer 62.

In some embodiments, the mixtures are heated and injected into their respective cold mold forms under high pressure. In some embodiments, the co-mold temperature reaches about room temperature. The molded pieces that emerges from the molds are known in the art as the “green part.”

Next, the polymeric binder is removed from the green part pieces. In some embodiments, the debinding process uses solvents, heat (thermal), or both. Debinding solvents and temperatures to remove the polymeric binder are known to those skilled in the art. In an illustrative embodiment, an example of a thermal temperature to debind the polymer binder is a temperature of less than about 300° C. In some embodiments, the debinding process takes place under an argon atmosphere.

After the debinding process, the “brown part” pieces are oriented to each other so that the articular layer 58 is coupled to the condylar surface 66 of the substrate 60 to form the femoral component 12. In some embodiments, the articular layer 58 is coupled to the condylar surface 66 of the substrate 60 to formal the femoral component 12, and the bone-engaging layer 62 is coupled to the bone-facing surface 64 of the substrate 60. The brown part pieces are sintered in a vacuum at about 1350° C. to about 1450° C. to bond the two pieces (the articular layer 58 and the substrate 60) or three pieces together generating a femoral component 12 comprising a bone-engaging layer 62. In some embodiments, this sintering process occurs for about 4 hours. Those skilled in the art will recognize that other amounts of time may be substituted. In some embodiments, the step of sintering occurs in a controlled environment of argon with an oxygen concentration of less than 50 ppm. After this step of sintering, the femoral component 12 or femoral component 12 including a bone-engaging layer 62 is released from the vacuum.

In some embodiments, the sintered piece generating the femoral component 12 comprises the substrate 60 and the articular layer 58, wherein the articular layer 58 has a thickness of about 2 mm to about 5 mm.

In some embodiments, the articular layer 58 of the femoral component 12 is mechanically polished to remove any excess material. In some embodiments, prior to the oxidizing step, the articular layer 58 is further processed by subjecting it to an abrasive surface preparation process that includes but is not limited to grinding, buffing, mass finishing, and vibratory finishing. The further processing step is used to induce an altered surface roughness to generate a more uniform oxidized outer surface.

Next, the femoral component 12 undergoes an oxidizing step to oxidize a portion of the articular layer 58. In some embodiments, an oxidizing process used is Kemp's process, wherein the femoral component 12 is heated to a temperature of about 500° C. to about 550° C. for about three hours to about five hours under an atmosphere of argon and oxygen in a fluidized bed. In some embodiments, the femoral component 12 is heated to between 550° C. to about 650° C. for about five hours to about six hours under an atmosphere of argon and oxygen in a fluidized bed. In some embodiments, the oxygen comprises about 1% to about 3% and argon comprises about 97% to about 99% of total gas volume in the fluidized bed. In some embodiments, the oxygen comprises about 2.5% of total gas volume in the fluidized bed. In some embodiments, inert oxide granules are used in the oxidizing process. In some embodiments, the inert oxide granules comprise ZrO₂. In some embodiments, the inert oxide granules comprise alumina oxide particles. For further disclosure on the step of oxidizing see U.S. Pat. Nos. 6,447,550 and 5,324,009 both of which are incorporated by reference in their entirety.

The step of oxidizing oxidizes a portion of the articular layer 58. The oxidized portion of the articular layer 58 comprises a layer of ZrO₂. In some embodiments, the ZrO₂ of the articular layer 58 has a thickness of about 4 μm to about 6 μm. In some embodiments, the ZrO₂ has a thickness of about 5 μm.

In some embodiments, the method of forming the femoral component 12 comprises further post-processing steps. In some embodiments, the method further includes post-polishing of the articular layer 58. The step of post-polishing is known to those skilled in the art. For example, the femoral component 12 is post polished by hand or by mass finishing in a rotating tumbler with polishing media.

In one illustrative example, the step of oxidizing can be done using a SCHWING Model HT1050-3560 Fluidized Bed System (fluidizing media; white aluminum oxide, 150 mesh, 99% pure) for surface oxidizing zirconium alloys in the form of discs, fatigue bars, and knee implants is as follows.

Lots of discs (qty. 68), fatigue bars (qty. 15), and knee implants (qty. 6) were processed using the aforementioned fluidizing bed to apply an even ZrO₂ layer onto the surface of each of the discs, bars, and implants. Note that some samples were washed using detergent, and all samples were wiped with acetone prior to processing. It was found that handling parts with latex gloves and wiping thoroughly with acetone yielded good results, allowing surfaces to be free of any oils or contaminants which may impact uniformity of the surface treatment. Samples were placed into a basket and secured using stainless steel wire. A fluidized bed was heated to the target temperature (about 500° C. or about 540° C.) using 100% air, then switched to an inert atmosphere during the final 30 minutes by using 100% argon as the fluidizing gas. Next, parts were deposited into the fluidized bed (under 100% argon) and allowed to heat up for approximately 15 minutes. Then a treatment atmosphere was introduced, containing a 2.5% oxygen mixture. Process time began once oxygen was introduced. Samples were allowed to process between 3 and 7 hours, then removed from the fluidized bed and allowed to air cool suspended in the fixture. Upon completion, parts appear gray-blue in color.

A summary of the parameters of the surface oxidation procedure is shown below in Table 1.

TABLE 1

| | | | O₂ Content | |
|---|---|---|---|---|
| | | Temperature | Gas Mixture | Treatment Time |
| Samples | | (° C.) | (%/mole of gas) | (hours) |
| 16 | discs | 540 | 2.5 | 3, 4, 5, 6 |
| 16 | discs | 500 | 2.5 | 3, 4, 5, 6, 7 |
| 14 | discs | 540 | 2.5 | 4 |
| 14 | discs | 500 | 2.5 | 5.5 |
| 7 | bars | | | |
| 4 | discs | 540 | 2.5 | 5 |
| 4 | bars | | | |
| 3 | knees | | | |
| 4 | discs | 540 | 2.5 | 5 |
| 4 | bars | | | |
| 3 | knees | | | |

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the method, apparatus, and system described herein. It will be noted that alternative embodiments of the method, apparatus, and system of the present disclosure may not include all of the features described yet still benefit from at least some of the advantages of such features. Those of ordinary skill in the art may readily devise their own implementations of the method, apparatus, and system that incorporate one or more of the features of the present invention and fall within the spirit and scope of the present disclosure as defined by the appended claims.

1. A knee prosthesis comprising:
a femoral component configured to be coupled to a distal end of a patient's femur, the femoral component comprising
(i) a substrate comprising a titanium alloy having (a) a condylar surface, and (b) a bone-facing surface opposite the condylar surface, and
(ii) an articular layer disposed on the condylar surface, the articular layer comprising an alloy of zirconium and niobium,
wherein a portion of the alloy of zirconium and niobium is oxidized zirconium, and
wherein the oxidized zirconium forms an outer surface of the femoral component.

2. The knee prosthesis of claim 1, further comprising a bone-engaging layer disposed on the bone-facing surface.

3. The knee prosthesis of claim 1, wherein the articular layer has a thickness of about 2 mm to about 3 mm.

4. The knee prosthesis of claim 1, wherein the articular layer comprises at least about 93% by weight zirconium.

5. The knee prosthesis of claim 1, wherein the articular layer comprises up to about 7% by weight niobium.

6. The knee prosthesis of claim 1, wherein the articular layer comprises about 97.5% by weight of zirconium and about 2.5% by weight of niobium.

7. The knee prosthesis of claim 1, wherein the oxidized zirconium has a thickness of about 3 μm to about 6 μm.

8. The knee prosthesis of claim 7, wherein the oxidized zirconium has a thickness of about 5 μm.

9. A method of forming a femoral component of a knee prosthesis, the method comprising the step of:
utilizing direct energy deposition to apply an articular layer to a condylar surface of a femoral component.

10. The method of claim 9, wherein the articular layer comprises an alloy of zirconium and niobium.

11. The method of claim 10 wherein the articular layer comprises about 97% by weight of zirconium and about 3% by weight niobium.

12. The method of claim 11 further comprising the step of oxidizing a portion of the articular layer.

13. The method of claim 12 wherein the articular layer has a thickness of about 2 mm to about 3 mm.

14. The method of claim 12, wherein the oxidized portion of the articular layer has a thickness of about 5 μm.

15. A method of forming a femoral component of a knee prosthesis, comprising:
injecting a first mixture comprising a zirconium alloy and a binder agent into a first mold to form an articular layer; and
injecting a second mixture comprising a titanium alloy and a binder agent into a second mold to form a substrate,
sintering the articular layer and substrate together, and oxidizing a portion of the articular layer.

16. The method of 15, wherein the first mixture comprises at least about 93% by weight zirconium and up to about 7% by weight niobium.

17. The method of claim 15 further comprising sintering a bone-engaging layer.

18. The method of claim 15, wherein the oxidizing step is performed in a fluidized bed comprising about 1% to about 3% oxygen and about 97% to about 99% argon.

19. The method of claim 18, wherein the oxidizing step generates a zirconium oxide layer having a thickness of about 2 μm to about 5 μm on a portion of the articular layer.
